(19) United States
(12) Patent Application Publication
(10) Pub. No.: US 2011/0059863 A1
(43) Pub. Date: Mar. 10, 2011

(54) IN VIVO EVOLUTION OF HYDROGENASES USING A HYDROGEN-SENSING SYSTEM

(75) Inventors: Maria Lucia Ghirardi, Lakewoood, CO (US); Matt Wecker, Boulder, CO (US); Matthew Posewitz, Golden, CO (US)

(73) Assignee: Alliance for Sustainable Energy, LLC, Golden, CO (US)

(21) Appl. No.: 12/918,182

(22) PCT Filed: Mar. 10, 2009
(86) PCT No.: PCT/US09/36669

§ 371 Date: Aug. 18, 2010

Related U.S. Application Data
(60) Provisional application No. 61/035,284, filed on Mar. 10, 2008.

(51) Int. Cl. C40B 30/08; C12Q 1/26; C12N 15/63; C12N 1/00

(52) U.S. Cl. 506/11; 435/25; 435/320.1; 435/243

(57) ABSTRACT

Provided herein are methods for measuring H2 production by a hydrogenase. Also provided are methods for evolving a hydrogenase or hydrogen-sensing system by comparing the level of production of H2 by the hydrogenase to the level Of H2 produced by a wild-type hydrogenase cultured under the same conditions and selecting a microorganism with increased H2 production over the H2 production by a microorganism having a wild-type hydrogenase. Further provided herein are microorganisms and plasmids comprising a hupSL promoter coupled with a reporter gene.

### CONTRACTUAL ORIGIN

The United States Government has rights in this invention under Contract No. DE-AC36-08G028308 between the United States Department of Energy and the Alliance for Sustainable Energy, LLC, manager and operator of the National Renewable Energy Laboratory.

### BACKGROUND

Increases in projected energy demand in conjunction with a decrease in fossil fuel reserves and the drive to reduce CO₂ emissions is stimulating the development of clean renewable energy technologies. Such clean technologies include wind, photovoltaics, solar, thermal, geothermal, hydroelectric, and biofuels energy sources. Biofuels include bioethanol, biodiesels, and biohydrogen, though the latter is considered an energy carrier rather than a fuel. Biohydrogen production using, for example, the green alga C. reinhardtii can potentially convert 10% of the incident solar energy into H₂ while minimizing production of toxic by-products or CO₂.

Photobiological H₂ production from water is a clean, non-polluting, and renewable technology showing promise in the future hydrogen economy, however, current systems exhibit limited efficiency relative to theoretical efficiency in conversion of light to H₂. For example, biological hydrogenases are sensitive to oxygen, the obligatory by-product of photosynthetic water oxidation, resulting in decreased H₂ production in the presence of increasing levels of oxygen. Further, availability of hydrogenase reductants necessary to drive H₂ production is low due to the existence of competing metabolic pathways.

The foregoing examples of the related art and limitations related therewith are intended to be illustrative and not exclusive. Other limitations of the related art will become apparent to those of skill in the art upon a reading of the specification and a study of the drawings.

### BRIEF DESCRIPTION OF THE DRAWINGS

FIG. 1 illustrates electron transport pathways leading to H₂ production from reduced ferredoxin.

FIG. 2 illustrates a H₂-sensing system of R. capsulatus. A. The hupT kinase and hupR response regulator are phosphorylated in the absence of H₂, leading to no transcription of the hupSL uptake hydrogenase. B. The presence of H₂ results in dephosphorylation of the kinase and the response regulator. The regulator then binds to the promoter site of the uptake hydrogenase hupSL and upregulates hupSL expression. This system, when coupled to a reporter gene such as, for example, Green Fluorescence Protein serves as a high-throughput assay for measuring hydrogenase H₂ production and a means of selecting cells with improved H₂ output.

FIG. 3, like FIG. 2, demonstrates a H₂-sensing system. FIG. 3 further illustrates the directed evolution of hydrogenases. Hydrogenase genes mutagenized and shuffled to provide a library of hydrogenases are examined for their level of H₂ output. The library can be iteratively shuffled in subsequent rounds of selection.

FIG. 4 demonstrates uses for a hydrogen-sensing system.

FIGS. 5A-D illustrate the production of an exemplary promoter/reporter gene construct, hupSL::lacZ, on a plasmid containing hydrogenase assembly proteins coupled to Tn5α promoters.

FIG. 6 illustrates the hupSL promoter region (SEQ ID NO:1). Elements of the promoter region are indicated as follows: the primary hupSL enhancer binding site is highlighted (gray box). The putative HupR enhancer site is underlined. Sequence protected from DNA digestion by HupR is double-underlined. The Integration Host Factor (IHF) binding site is in lowercase. The −35 and −10 Sigma 70 sites are in lowercase bold. The transcription initiation site is in lowercase underlined. The HupSL protein start site is the last three bases. The reporter gene (not shown) is fused, in-frame downstream from the HupSL protein start site. Randomization can be concentrated between the artificial BspE1 restriction site and the neighboring MfeI restriction site (both uppercase italic).

Exemplary embodiments are illustrated in referenced figures of the drawings. It is intended that the embodiments and figures disclosed herein are to be considered illustrative rather than limiting.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Embodiments herein provide a method for improving the signal:noise ratio of the hydrogen-sensing system through randomization and selection of one or any combination of the following: the hydrogen sensing protein HupUV, the histidine kinase HupT, the hydrogen transcription regulator protein HupR, and the HupSL promoter.

Embodiments herein provide a biologically-based assay to screen large numbers of hydrogenases for improved H₂ production properties.

Further embodiments provide a biologically-based assay to screen libraries of mutagenized hydrogenase genes for improved H₂ production properties.

Still further embodiments provide methods for measuring H₂ production, methods of evolving a hydrogenase, and methods for evolving a H₂-sensing system.

Other embodiments provide methods for screening or selecting for an oxygen-resistant and/or carbon monoxide resistant hydrogenase.

In addition to the exemplary aspects and embodiments described above, further aspects and embodiments will become apparent by reference to the drawings and by study of the following descriptions.

### DETAILED DESCRIPTION

Systems are provided for a high-throughput screen based on the H₂-sensing properties of particular proteins present in photosynthetic bacteria. In some aspects, this screen may be used to direct the evolution of the hydrogen sensing system itself to increase its sensitivity, and/or specificity to H₂, particularly in the presence of O₂ and/or CO. In other aspects, this screen is used to direct the evolution of [FeFe]-hydrogenases to further increase their O₂ tolerance. In other aspects this screen is used to identify hydrogenases tolerant to O₂ and/or CO. In still further aspects this screen is used to measure hydrogenase activity.

In some embodiments, the hydrogenases and the sensing system are derived from nature. In other embodiments, the sequence encoding the hydrogenase, the hydrogen assembly proteins, and/or the Hydrogen-sensing system is shuffled or otherwise mutated relative to the wild-type sequences.

Systems, methods, and assays described herein provide a significant advantage in that measurement of H₂ production does not require destruction of the host microorganism. In some aspects, measurement of H₂ production permits continuous selection of host microorganisms having improved hydrogenase characteristics over wild-type hydrogenases. Measurement of H₂ production is achieved through activation of an H₂ sensor in response to the presence of H₂.

### DEFINITIONS

The following definitions are provided to facilitate understanding of certain terms.

“Expression” refers to transcription and translation occurring within a host cell. The level of expression of a DNA molecule in a host cell may be determined on the basis of either the amount or corresponding mRNA that is present within the cell or the amount of DNA molecule encoded protein produced by the host cell.

The phrase “foreign DNA” refers to any DNA transferred from foreign origin. Exemplary foreign DNAs include but are not limited to DNA from foreign species, recombinant DNA, mutagenized DNA, shuffled DNA, etc. Foreign DNA can be transferred in many ways known to those skilled in the art, including, for example, in the form of a plasmid, cosmid, insertion element, transposon, chromosome, or naked DNA such as in homologous recombination.

“Host microorganism” refers to a microorganism useful for the expression of proteins encoded by foreign DNA or other low molecular weight nucleic acid.

“Hydrogenase” refers to any enzyme than can either produce or utilize hydrogen. In some instances, a nitrogenase can be considered a hydrogenase.

“Oxygen-resistant” refers to any measurable decrease in oxygen sensitivity in a hydrogenase as compared to a hydrogenase having a reference oxygen sensitivity, for example, as compared to a wild-type hydrogenase from which an oxygen-resistant enzyme has been made.

“Oxygen-sensitive” refers to the wild-type or reference oxygen sensitivity found in a native hydrogenase.

“Plasmid” refers to an extrachromosomal, circular DNA molecule capable of replication in bacteria. When the word plasmid is used herein, it is understood that any other foreign DNA can be substituted.

“Promoter” refers to the region of DNA at the upstream (5-prime) end of a gene or operon that serves as the initiation site for transcription.

“Reporter gene” refers to a gene encoding a product that can readily be measured, such as a fluorescing protein.

“Wild-type” refers to the typical form of an organism, strain, gene, or characteristic as it occurs in nature.

### H₂-Hydrogenases

Exemplary hydrogenases useful herein include [Fe—Fe] hydrogenases, [Ni—Fe] hydrogenase, and Fe—S free hydrogenases.

In general, [Fe—Fe]-hydrogenase enzymes characteristically possess a catalytic site consisting of a bimetallic center containing two Fe and two S atoms (2Fe2S center), bridged by cysteinyl sulfur to an electron relay [4Fe4S] center (4Fe4Scenter). The iron atoms of the catalytic 2Fe2S center are joined together by a combination of organic, sulfur, and carbon monoxide ligands. The chemistry of the [Fe—Fe]-hydrogenase catalytic core is reactive with respect to hydrogen, typically possessing very high hydrogen-production and/or oxidation rates. However, this same catalytic core is also highly sensitive to inactivation by oxygen. As a protective measure against inactivation by oxygen or other like molecules, the catalytic core is typically buried deep within the protein, where access to the core is limited. As a result, interface of the hydrogenase catalytic site with surface surroundings is principally limited to two channels which direct diffusion of synthesized hydrogen from the enzyme interior to the external environment. The channels are also the primary access routes of oxygen to the metallo-catalytic site within hydrogenase enzyme. Reverse diffusion of the oxygen from the surface of the enzyme into the channel and on to the active site, allows oxygen to bind to the 2Fe2S-center, inactivating the enzyme. Under normal physiologic conditions this represents a fairly normal inhibitory response for the hydrogenase enzyme, however, under the artificial conditions of expressing bulk amounts of H₂, this is a fairly major limitation.

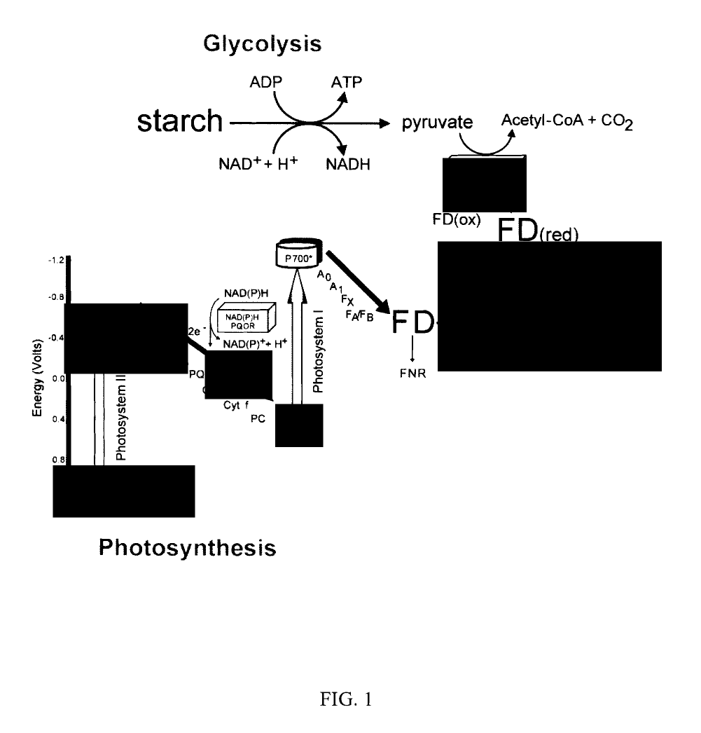

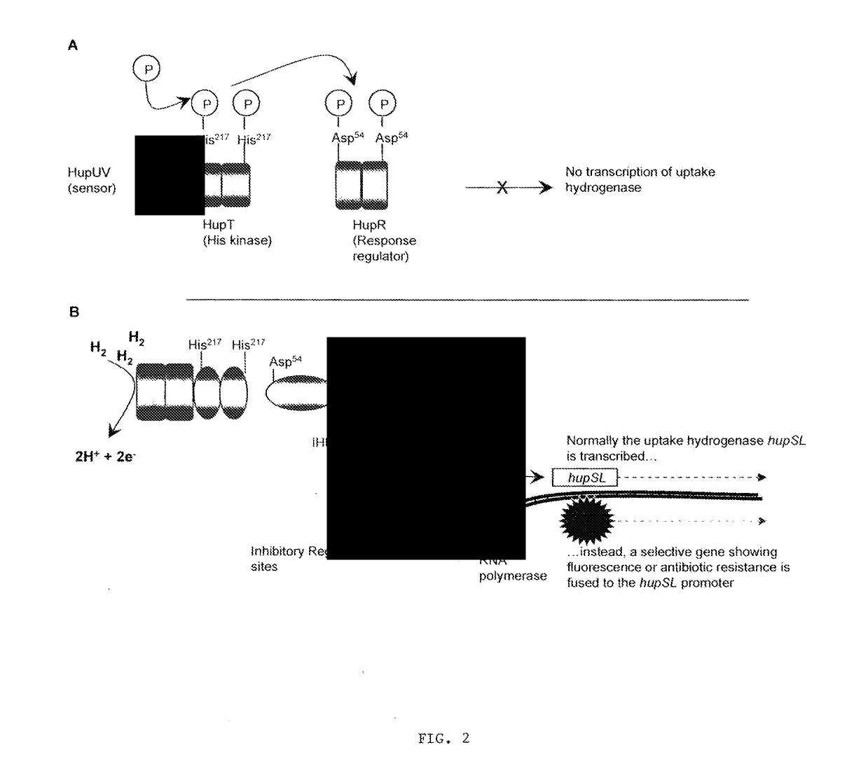

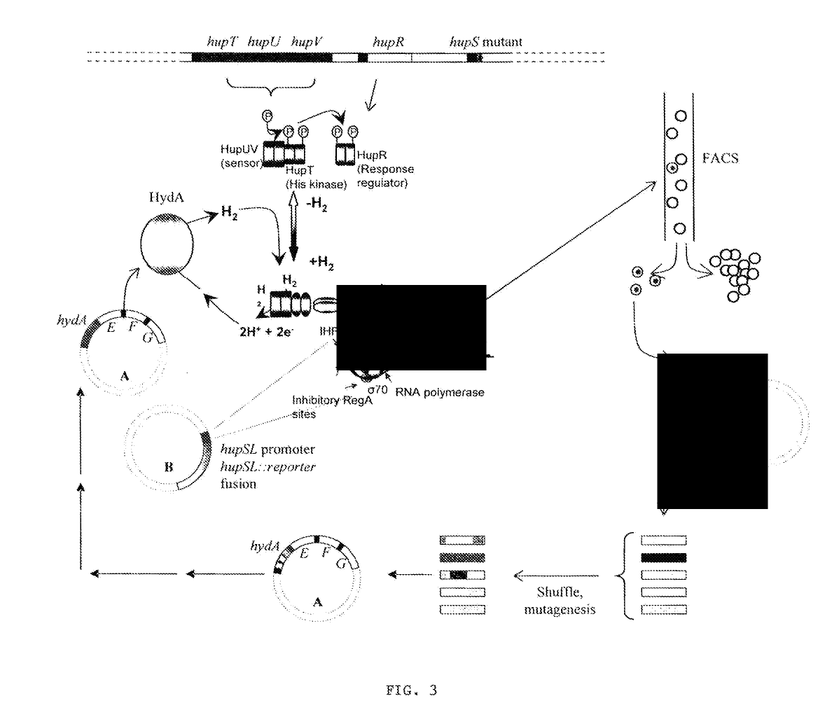

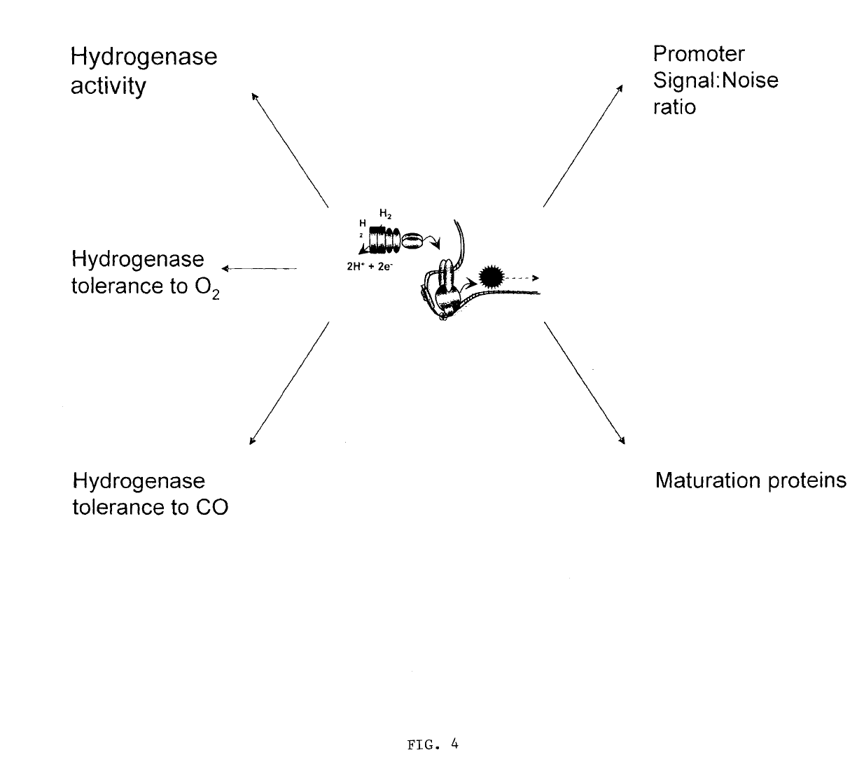

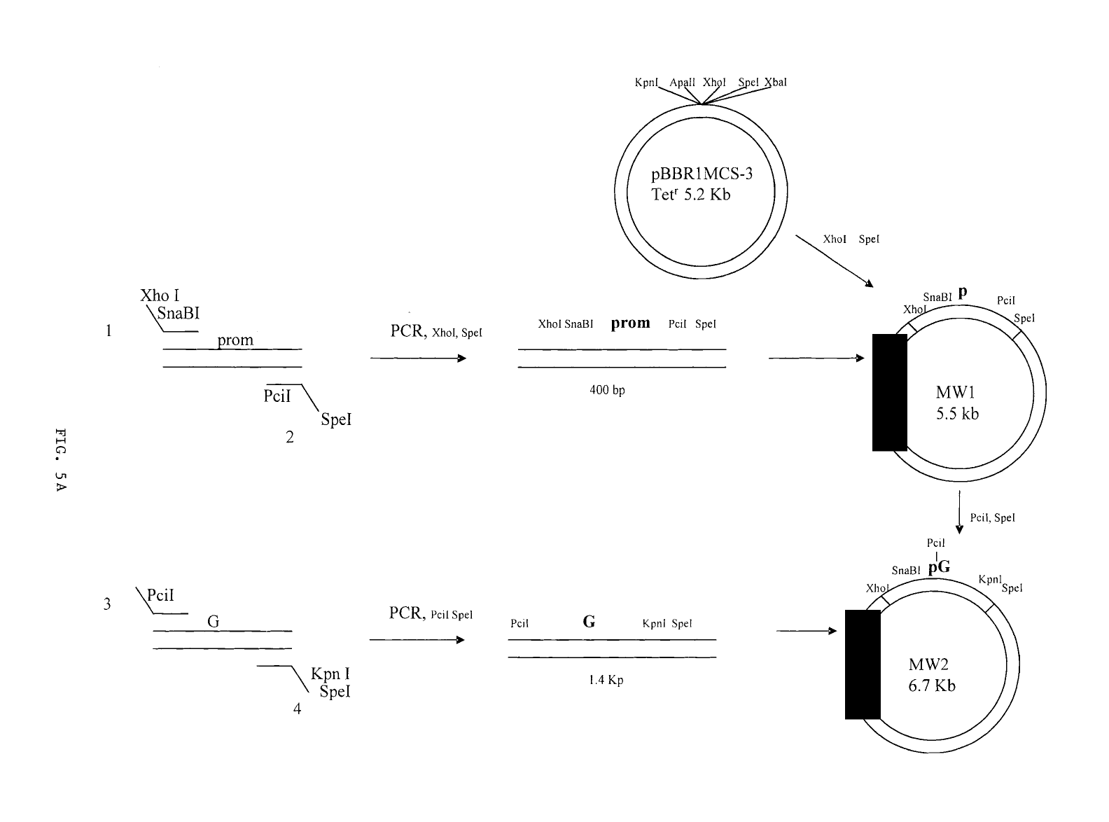

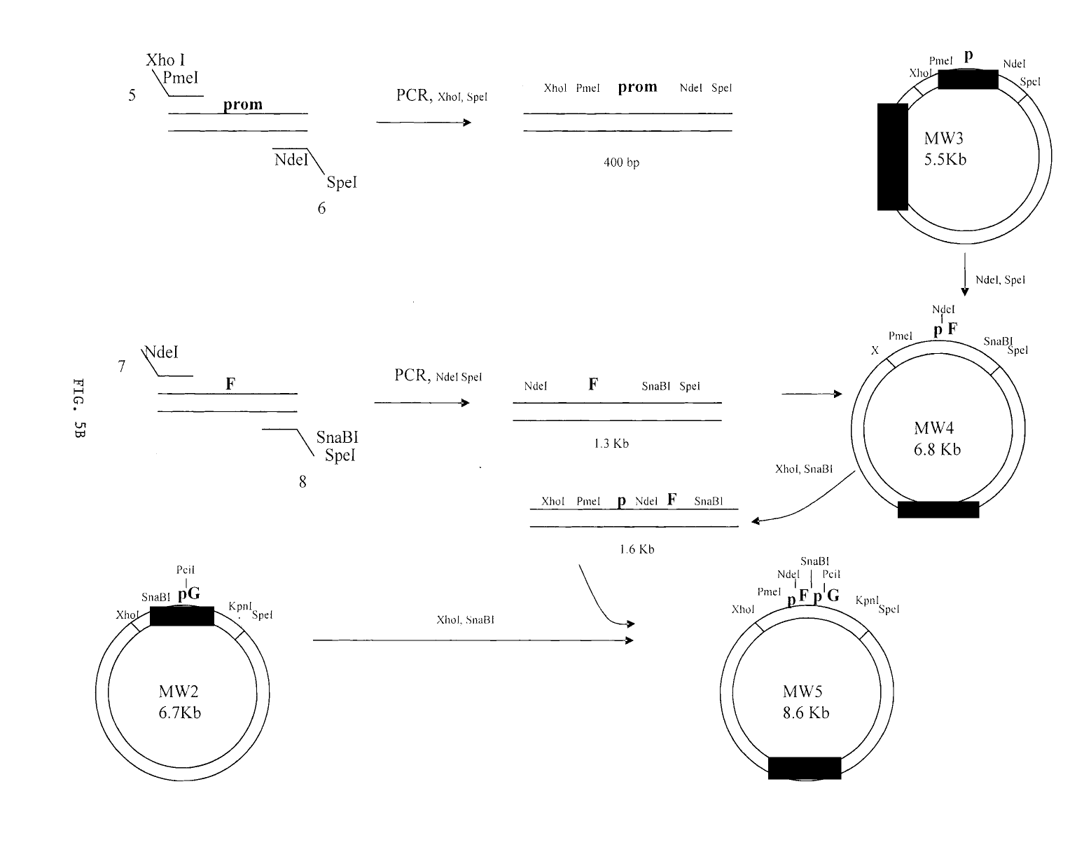

Some microalgae and cyanobacteria are capable of photoproduction of H₂ gas using water as the only electron donor. The energy generating reactions of photosynthesis couple water oxidation directly to H₂ production through the activity of hydrogenase enzymes rather than to CO₂ fixation into sugars (See FIG. 1).

As illustrated in FIG. 1, there are three distinct H₂-production pathways using low-potential electrons provided either by carbon fermentation or by light-driven photosynthetic pathways: two H₂-photoproduction pathways and a dark, fermentative H₂-production pathway. The first of the H₂-photoproduction pathways (also termed direct biophotolysis) is dependent on the activities of both Photosystem II (PSII) and Photosystem I (PSI). It involves light-induced water oxidation at PSII, transfer of electrons from PSII to PSI, and light-dependent re-energizing of electrons by PSI to reduce ferredoxin (FD), the physiological electron donor to C. reinhardtii hydrogenases. The second H₂-photoproduction pathway involves the non-photochemical reduction of plastoquinone (PQ) using electrons from NAD(P)H, generated primarily from starch catabolism in the chloroplast, followed by light-dependent reduction of FD by PSI. This pathway is independent of PSII but it is dependent on NAD(P)H-plastoquinone oxidoreductase (NPQR). The dark, fermentative H₂-production pathway in C. reinhardtii is coupled to starch catabolism. In all pathways, FD is the direct electron donor to hydrogenase.

A commercial and cost-effective H₂-producing system can address one or more of the following issues: (a) competition for photosynthetically-generated reductants among different pathways; (b) limitations on the rate of electron transport and the predominance of cyclic electron transfer under H₂-producing conditions; and, (c) low sunlight-conversion efficiency of H₂ photoproduction due to the presence of large light-absorbing pigments.

### Oxygen Sensitivity of [FeFe]-Hydrogenases

Algal hydrogenases belong to the class of [FeFe]-hydrogenases that are characterized by the presence of an H-cluster at their catalytic centers. The H-cluster structure endows [FeFe]-hydrogenases with the highest catalytic turnover number of all hydrogenases (6000-9000 s⁻¹), but these enzymes have extreme sensitivity to O₂ inactivation. This prevents sustained H₂ production from occurring, unless O₂ is removed from the medium. Oxygen inhibition occurs by the irreversible binding and oxidation of the distal iron in the [2Fe-2S] cluster by O₂.

The green alga C. reinhardtii contains two nuclear-encoded [FeFe]-hydrogenases, HYDA1 and HYDA2. Both hydrogenase genes are upregulated under anaerobic conditions and their gene products are capable of catalyzing H₂ production. Active [FeFe]-hydrogenases from C. reinhardtii and Clostridium acetobutylicum have been heterologously expressed in E. coli with their respective assembly proteins or with the assembly proteins from C. acetobutylicum. Researchers at the National Renewable Energy Laboratory have identified factors that confer O₂-tolerance to [FeFe]-hydrogenases, such as accessibility of O₂ to the enzyme catalytic site, and are attempting to engineer enzymes that function under aerobic conditions using rational, site-directed mutagenesis to generate O₂ tolerant [Fe—Fe]-hydrogenases. This rational approach is based on the investigation of gas channels present in the structure of hydrogenases, followed by mutagenesis efforts aimed at closing these channels. However, this approach requires detailed knowledge of the structure of the enzyme, the use of molecular dynamics simulations to identify pathways for gas diffusion through the structure, and is fairly time consuming as each potential mutant needs to be separately created, purified and tested.

An alternative means of creating O₂ tolerance in hydrogenases is to evolve the enzyme using gene shuffling, a random genetic approach used successfully to create diversity in catalysts and to yield proteins with new functionality. Nagy et al. (Application of gene-shuffling for the rapid generation of novel [FeFe]-hydrogenase libraries. Biotechnol Lett, 2007. 29(3): p. 421-30), the contents of which are incorporated herein by reference, describe the expression of active, shuffled [Fe—Fe]-hydrogenases in a heterologous E. coli-expression system. However, the lack of appropriate high-throughput assays prevents the sampling of large, recombinant populations of hydrogenases for improved activity. One assay purportedly useful for mid-throughput screening uses the ability of H₂ to sensitize a palladium/tungsten oxide film, however, technical issues have prevented its use in high-throughput mode.

### H₂-Sensing System

The only known H₂-sensing systems in nature are those occurring in nitrogenase-containing photosynthetic bacteria, such as Rhodobacter capsulatus, Rhodobacter palustris, Wautersia eutropha (Ralstonia eutropha) and Bradyrhizobium japonicum. The H₂-sensing system acts to upregulate expression of the cell's uptake hydrogenase in response to exogenous H₂ or H₂ generated by the cell's own nitrogenase (FIG. 2). The four characterized H₂-sensing systems are nearly identical. Shown in FIG. 2 is the H₂-sensing system of R. capsulatus. The principal system components are a H₂ sensor protein (HupUV), a histidine kinase (HupT), a transcription regulator (HupR), and an uptake hydrogenase (HupSL). In the absence of H₂, the sensor HupUV interacts with the kinase HupT leading to its autophosphorylation. The activated kinase then phosphorylates the HupR regulator. Phosphorylated HupR no longer associates with the hupSL upstream promoter region, leading to down-regulation of uptake hydrogenase expression. In the presence of H₂, the kinase and the regulator proteins are dephosphorylated, and the HupR regulator binds to and activates σ70 RNA polymerase (RNAP)-dependent transcription of hupSL encoding the uptake hydrogenase. The hupSL promoter region has an integration host factor (IHF) site that, when bound by IHF, increases interaction of the HupR regulator with RNAP during the activation of hupSL. The hupSL promoter has two predicted RegA binding sites that mediate negative redox control of hupSL by interfering with the binding of IHF and RNAP, leading to a 3 to 6-fold reduction in hydrogenase activity. The regulator HupR is constitutively expressed at low levels in R. capsulatus. Both hupUV and hupT are transcriptionally regulated from the hupT promoter and are transcribed at levels 50-fold lower than hupR.

The signal from H₂-sensing systems has been coupled to β-galactosidase transcription in R. capsulatus and R. palustris. In R. capsulatus, a promoterless lacZ gene was fused to a truncated hupS gene downstream of the hupR activation region (hupS::LacZ). The hupS::lacZ reporter system responded to the presence of H₂ by producing the enzyme β-galactosidase. β-galactosidase levels were measured by its hydrolysis of o-nitro-phenyl-β-D-galactopyranoside (ONPG) to the indicator o-nitrophenol. β-galactosidase levels of 20-fold over background were noted in the presence of H₂, whether the H₂ was produced by nitrogenases under N₂-fixing conditions or was exogenously supplied (Colbeau, A., and P. M. Vignais, Use of hupS::lacZ gene fusion to study regulation of hydrogenase expression in Rhodobacter capsulatus: stimulation by H2. J Bacteriol, 1992. 174(13): p. 4258-64). Similarly, in R. palustris, lacZ expression was up-regulated 10-fold when the cells were grown under photoheterotrophic N₂ fixing (H₂ evolving) conditions as compared to conditions under which N₂ was not fixed (Rey et al., Regulation of uptake hydrogenase and effects of hydrogen utilization on gene expression in Rhodopseudomonas palustris. J Bacteriol, 2006. 188(17): p. 6143-52). The hupS::lacZ system proved useful in understanding the mechanism of the H₂-sensing system and in detailing hupSL expression levels in a number of strains, including mutations in hupU, hupV, hupT, hupR, and mutants lacking nitrogenases. The system was also used to define elements of the hupSL promoter region in R. capsulatus. While the hupS::lacZ system was valuable for analyzing mechanisms of H₂ sensing itself, the method of sensing the signal destroyed the cells themselves, which limits use of the system for efficient high-throughput evolution of hydrogenases. Furthermore, given that β-galactosidase levels of 20-fold over background were noted, this signal-to-noise ratio of 20 would indicate that a large number of iterative cyclings would be necessary in order to parse through a population of moderate size such as 1 million cells. The primary problem is that cells having little H₂ production will not be efficiently separated from cells having greater H₂ production, leading to failure of enrichment.

The H₂-sensing protein HupUV in R. capsulatus as well as its homologue in W. eutropha are O₂ tolerant. The ability to assay for H₂ production under partially aerobic conditions by an in vivo genetic system and to couple the assay to cell selection has unexpectedly allowed for the screening of active hydrogenases and the directed evolution of hydrogenases with novel characteristics such as improved tolerance to O₂ and/or carbon monoxide.

Embodiments provided herein include a method for measuring H₂ production by a hydrogenase. The method comprises providing a foreign DNA comprising a hupSL or other like promoter coupled to a reporter gene; transferring the foreign DNA to a host microorganism lacking endogenous hydrogenase activity; transferring to the host microorganism one or more plasmids comprising genes encoding a recombinant hydrogenase; culturing the host microorganisms under conditions permissive to production of H₂ from the hydrogenase; and, measuring the level of the reporter gene product in vivo. In this method, production of H₂ by the hydrogenase activates the hupSL or other like promoter and the reporter gene product increases. This allows in vivo measurement of H₂ production by measuring the level of reporter gene product in vivo. Further embodiments include selection or enrichment of host microorganisms that show H₂ production.

In some aspects, the hydrogen-sensing system itself may be selected for specificity to H₂. In further aspects, the hydrogen-sensing system in part or in whole may be modified by nucleotide randomization and/or shuffling and may be selected and evolved for an increased signal-to-noise ratio in response to H₂. In further aspects the hydrogen-sensing system may be selected and evolved for an increased signal-to-noise ratio in the presence of O₂ or other environmental stimulus that would normally inhibit the discrimination of H₂ by the H₂-sensing system.

In some aspects the host organism itself may be mutagenized, or mutagenesis may be directed at specific proteins or protein complexes within the host organism, such as nitrogenases, or photosystem components, or other exogenous proteins may be added so as to direct H₂ production by the organism. In this way H₂ production can be monitored, and the host organism itself, and/or specific proteins can be selected and evolved to optimize H₂ production.

In some aspects, a pool of host microorganisms is provided with a library of recombinant hydrogenases such that each individual organism effectively has one or more unique hydrogenases. In further aspects, the host microorganisms are provided with associated assembly proteins or other proteins for the hydrogenase. Associated assembly proteins or hydrogenase support proteins include but are not limited to any proteins useful in assembling the hydrogenase or regulating its activity.

The reporter gene product can be any readily measured product, for example, antibiotic resistance or fluorescence. Exemplary fluorescence reporter genes include but are not limited to Green Fluorescent Protein, HaloTag, and SNAP. Reporter genes can confer resistance to any antibiotic, including but not limited to kanamycin, tetracycline, gentamicin, or spectinomycin. In certain embodiments, the reporter gene may be measurable without killing the microorganism (i.e., the reporter gene may be detected in a live microorganism, employing techniques that do not result in the death or destruction of the microorganism).

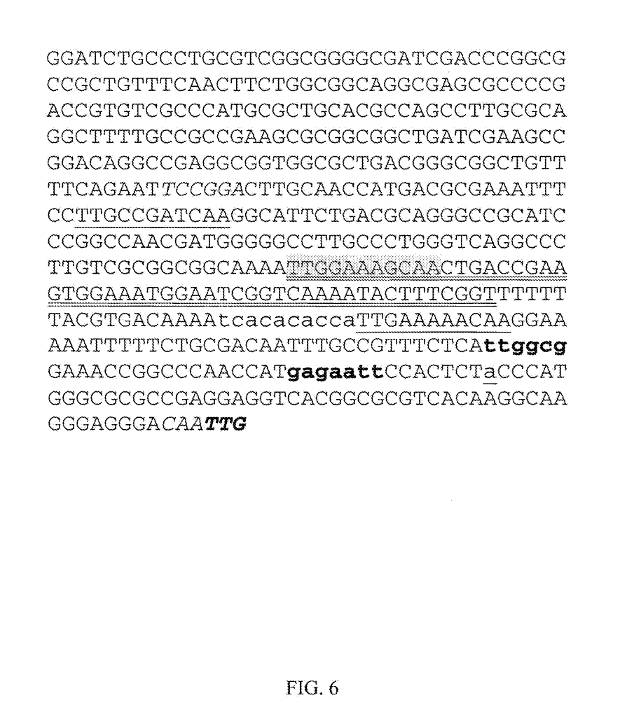

The sequence of the hupSL promoter region from R. capsulatus (represented by SEQ ID NO:1), along with elements contained therein, is illustrated in FIG. 6. Additional information on the promoter and its features can be found in Toussaint et al. Molecular Microbiology (1997) 26(5), 927-937, the contents of which are incorporated herein by reference. The hupSL promoter region from other species have been sequenced and can be used in various embodiments. One of skill in the art is able to locate these sequences in the literature. In some embodiments, the hupSL promoter region may be a fragment of SEQ ID NO:1 capable of initiating gene transcription (e.g., when fused to a reporter gene construct).

The hupSL promoter is illustrative of the class of promoters utilized by H₂ sensing systems. However, it will be understood herein that any promoter responsive to an H₂ sensing system can be substituted for the hupSL promoter.

In vivo measurement of the reporter gene product allows selection of intact microorganisms having hydrogenases with desired characteristics. Such characteristics can include increased hydrogen production, oxygen resistance, and/or carbon monoxide resistance. Illustratively, a microorganism with an antibiotic resistance reporter gene when grown under appropriate conditions on media containing the particular antibiotic will exhibit antibiotic resistance if the recombinant hydrogenase produces sufficient hydrogen such that the hupSL promoter is activated and the antibiotic resistance gene product is produced. Likewise, a microorganism with a fluorescent reporter gene when grown under appropriate conditions will exhibit fluorescence if the recombinant hydrogenase produces sufficient hydrogen such that the hupSL promoter is activated and the fluorescence gene product is produced. Fluorescence can be measured any number of ways known to those skilled in the art, for example, by a fluorescent plate reader or Fluorescently Activated Cell sorting (FACS). Identification of a desirable hydrogenase using such methods permits further modifications of the hydrogenase or use of the hydrogenase in hydrogen production.

In some embodiments, the host microorganism is R. capsulatus. In other embodiments, the host microorganism is W. eutropha. In still other embodiments, the host microorganism is R. palustris. In still other embodiments, the host microorganism is E. coli with a recombinant H₂-sensing system. The host microorganism generally lacks endogenous hydrogenase activity. In some aspects, the host microorganism comprises a disrupted, inactive uptake hydrogenase.

In some embodiments, the host microorganism comprises one or more plasmids. One such plasmid can comprise one or more recombinant hydrogenase genes which encode for the recombinant hydrogenases. In some aspects, the hydrogenase is obtained from other microorganisms. In other aspects, the hydrogenase is generated by mutagenizing and shuffling hydrogenase genes to provide a library of hydrogenases. WO 2006/093998, incorporated by reference herein in its entirety, describes an exemplary process for expression of hydrogenases in a host microorganism lacking endogenous hydrogenase activity.

The host microorganism can be cultured under conditions conducive or permissive to hydrogen production. Exemplary growth conditions are described in the Examples, though it will be understood that deviations or optimizations of growth conditions are contemplated herein. For example, growth conditions for one host microorganism can differ from the growth conditions for another host microorganism. Similarly, growth conditions can change with respect to the type of hydrogenase selected for, i.e. a carbon monoxide-resistant hydrogenase or an oxygen-resistant hydrogenase. Similarly, growth conditions can be changed to limit hydrogen production by the native nitrogenases, i.e. by supplying fixed nitrogen to the culture medium.

The host microorganism can be carried through one or more iterations of the process. By example, a pool of organisms resulting from one cycle of selection through the process, representing a pool of hydrogenases having enhanced hydrogenase activity, can have their representative pool of hydrogenases re-mutagenization and/or re-shuffled, inserted back into the host organism, and carried through one or more subsequent rounds of selection as necessary.

Thus, certain embodiments include a method of identifying an oxygen-resistant hydrogenase and/or a carbon monoxide resistant hydrogenase. The method comprises providing a foreign DNA comprising a hupSL or other like promoter coupled with a reporter gene; transferring the foreign DNA to a host microorganism lacking endogenous hydrogenase activity and comprising a recombinant hydrogenase; culturing the host microorganism in the presence of oxygen levels and/or carbon monoxide levels inhibitory to H₂ production by a wild-type hydrogenase; measuring the level of the reporter gene product in vivo; and comparing the level of production of H₂ by recombinant hydrogenase to the level of H₂ produced by the wild-type hydrogenase cultured under the same conditions. The production of H₂ by the hydrogenase activates the hupSL or other like promoter and the reporter gene product increases. The recombinant hydrogenase is oxygen-resistant if the level of H₂ produced is greater than that produced by the wild-type hydrogenase when both microorganisms are cultured in the presence of inhibitory levels of oxygen. Likewise, the recombinant hydrogenase is carbon monoxide-resistant if the level of H₂ produced is greater than that produced by the wild-type hydrogenase when both microorganisms are cultured in the presence of inhibitory levels of carbon monoxide.

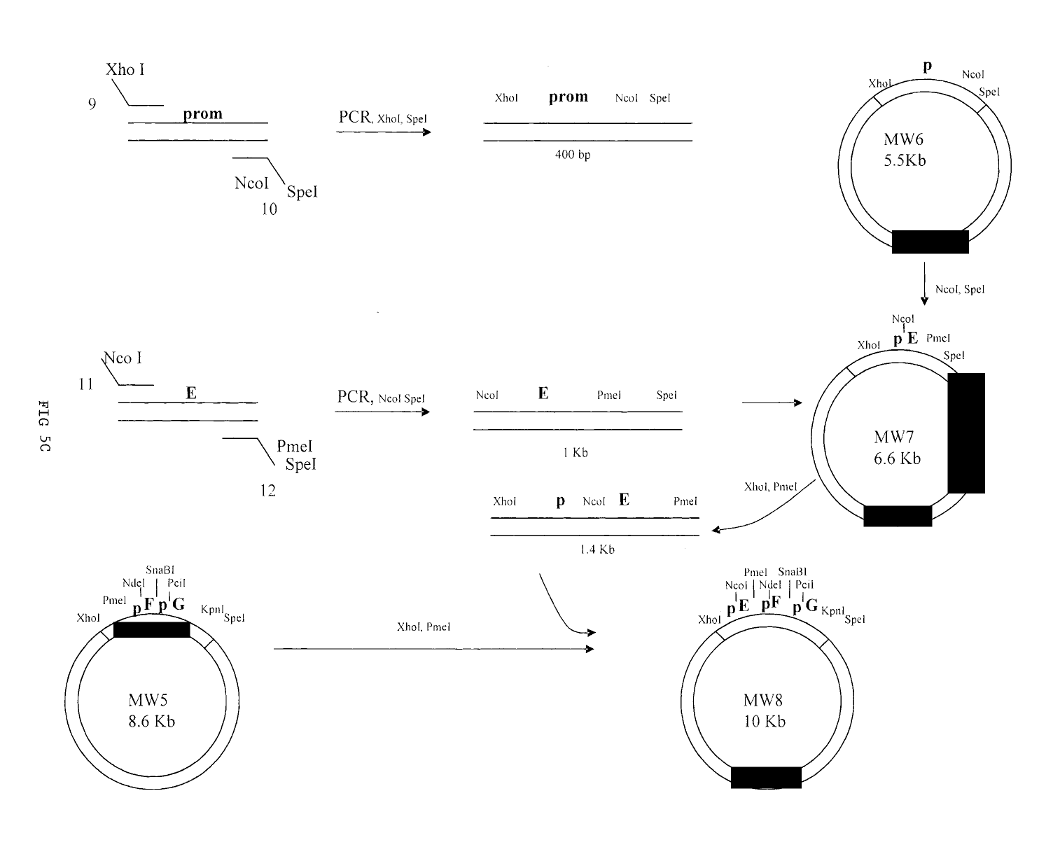

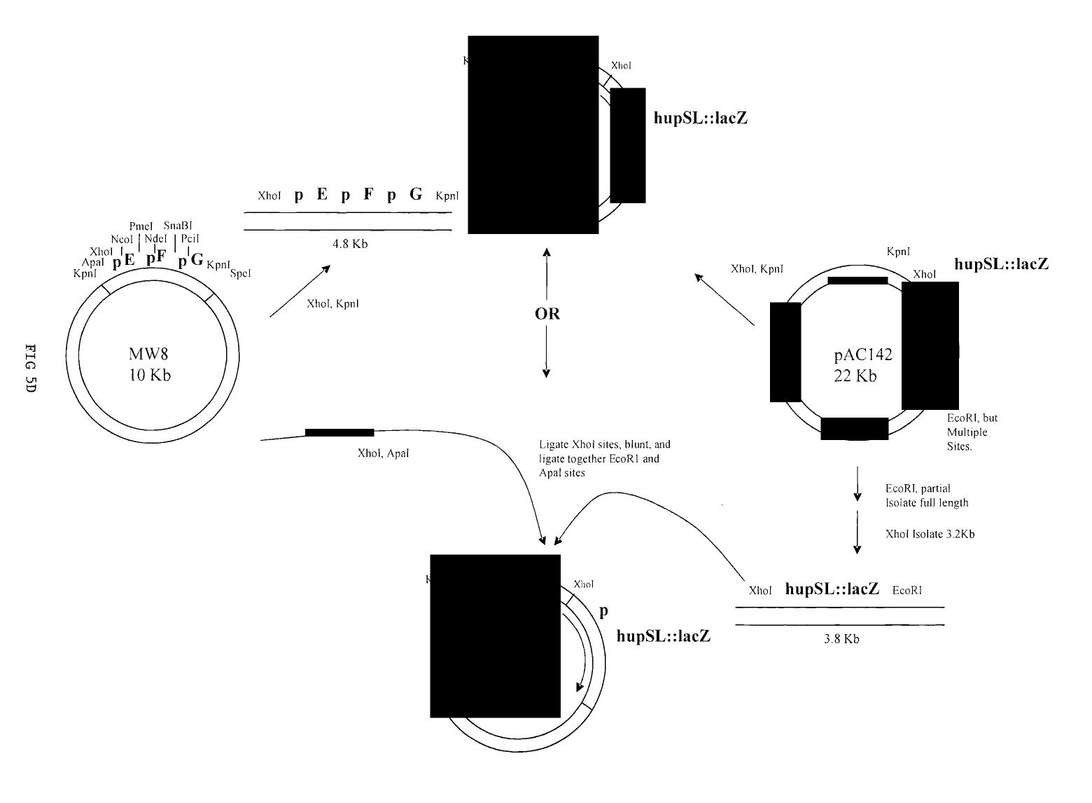

Embodiments described herein include a plasmid comprising a HupSL promoter coupled with a reporter gene, where the promoter may be evolved for H₂ specificity. Exemplary plasmid constructs are described in FIGS. 5A-D. Exemplary reporter genes are as described above. The plasmid can be transferred to host microorganisms and used according to the methods described herein.

Other embodiments include a microorganism comprising a foreign DNA which comprises a HupSL promoter coupled with a reporter gene and one or more plasmids comprising genes encoding one or more recombinant hydrogenases. In some aspects, the microorganism lacks endogenous hydrogenase activity. Plasmids can, for example, comprise one or more recombinant hydrogenase genes. In still further aspects, the microorganism comprises several plasmids encoding recombinant hydrogenase genes. The hydrogenase genes can be under the influence of one promoter, or can each be under the influence of their own promoter. In other aspects the microorganism comprises one or more plasmids comprising genes encoding hydrogenase assembly proteins, ferredoxin, and/or one or more proteins involved in the H₂-sensing apparatus.

Embodiments described herein include a plasmid comprising genes encoding associated proteins such as hydrogenase assembly proteins and/or proteins such as ferredoxin and/or proteins involved in the H₂-sensing apparatus. In some aspects, these associated proteins are obtained from other microorganisms. In other aspects the associated proteins are generated by mutagenizing and shuffling the associated proteins. In other aspects, these mutagenized and shuffled associated proteins are evolved for their ability to promote H₂ production as sensed and selected by the H₂-sensing system. In other aspects, these evolved associated proteins are utilized for the evolution of the hydrogenase in further selections. In yet other aspects, the associated proteins and the hydrogenase are co-evolved by mutagenesis and shuffling followed by selection for increased H₂ production using the H₂-sensing system.

By example, FIGS. 5A-D describe a method by which each hydrogenase assembly protein is coupled to a Tn5α promoter, and positioned on a plasmid containing the HupSL::lacZ reporter molecule. This example is illustrative of one approach to bringing some aspects of the method to practice. It will be understood by those of skill in the art that other approaches are readily available and contemplated herein.

Further embodiments include a high-throughput assay for measuring in vivo H₂ production by a recombinant hydrogenase. The assay comprises a host microorganism, an H₂ sensor, a recombinant hydrogenase, and a HupSL or other like promoter coupled with a reporter gene. Typically, the host microorganism lacks endogenous hydrogenase activity. In some aspects, the H₂ sensor is HupUV and can be endogenous to the host microorganism. The host microorganism can be cultured under oxygen or carbon monoxide levels inhibitory to H₂ production by a wild-type hydrogenase.

By example, FIG. 2 indicates the H₂-sensor system of R. capsulatus comprising a HupUV hydrogen sensor, a HupT kinase which is activated by HupUV in the absence of H₂, HupR, a response regulatory protein which is activated by HupT in the presence of H₂, the HupSL promoter which is upregulated by HupR in the presence of H₂ and a reporter gene that in the presence of H₂ is transcribed.

### Hydrogenase Evolution

In another embodiment a method is provided for evolving a hydrogenase. As described above, the method comprises providing a foreign DNA comprising a HupSL or other like promoter coupled to a reporter gene; transferring the foreign DNA to a host microorganism lacking endogenous hydrogenase activity; transferring to the host microorganism one or more plasmids comprising genes encoding a recombinant hydrogenase; culturing the host microorganisms under conditions permissive to production of H₂ from the hydrogenase; and measuring the level of the reporter gene product in vivo. The level of reporter gene product directly correlates to H₂ production. The level of production of H₂ by the recombinant hydrogenase can be compared to the level of H₂ produced by a wild-type hydrogenase having cultured under the same conditions. If the level of H₂ produced by the recombinant hydrogenase is greater than that produced by the wild-type hydrogenase the recombinant hydrogenase is considered “evolved”. The method can further comprise selecting a microorganism with increased H₂ production over the H₂ production by a microorganism having the wild-type hydrogenase.

Further embodiments include methods of evolving a hydrogenase comprising:
(a) providing a library of recombinant hydrogenases;
(b) transferring the recombinant hydrogenases to a pool of host microorganisms such that each host microorganism comprises one or more unique recombinant hydrogenases, each host microorganism further comprising hydrogenase support proteins, an H₂ sensor, and a hupSL promoter coupled with a reporter gene;
(c) culturing each of the host microorganisms under conditions permissive to production of H₂ from the recombinant hydrogenases;
(d) measuring the level of the reporter gene product in vivo produced by each microorganism;
(e) comparing the level of production of H₂ by each recombinant hydrogenase to the level of H₂ produced by a wild-type hydrogenase cultured under the same conditions; and
(f) selecting a microorganism with increased H₂ production over the H₂ production by a microorganism having a wild-type hydrogenase;
(g) isolating the plasmid comprising the recombinant hydrogenase from the selected microorganism;
(h) randomizing and/or shuffling the hydrogenase to generate a further library of recombinant hydrogenases; and
(i) iterating as necessary steps (b) through (h) and stopping at step (g) when the recombinant hydrogenase fails to demonstrate further increased H₂ production above the prior iteration;
wherein production of H₂ by the hydrogenase activates the hupSL promoter and the reporter gene product increases.

The steps of the above embodiment and any other embodiment or aspect described herein are not listed in any particular order and are not restricted to any particular order. Those skilled in the art understand that protocols as described herein can be optimized simply by reordering the steps.

When comparing hydrogenase activity, and in particular, comparing wild-type hydrogenase activity to recombinant hydrogenase activity, it is important that the host microorganisms are cultured under the same or similar conditions. For example, the microorganisms should be in similar growth phases, cultured under anaerobic conditions with the same media.

In another embodiment a method is provided for evolving the HupSL promoter region, and/or other components of the H₂-sensing system to increase the signal-to-noise ratio of the system in response to the presence of hydrogen. By example, the method comprises a library of partially randomized HupSL promoters inserted into the host organism creating a pool of cells each having a partially randomized HupSL promoter. Cells (“response negative” cells) are selected from the pool that in the absence of H₂ show very low expression of the reporter gene as compared to wild type cells containing non-randomized HupSL promoters. A second selection is performed upon those selected “response negative” cells where cells (“response positive”) are identified from this secondary pool which in the presence of H₂ show very high expression of the reporter gene as compared to wild type cells containing non-randomized HupSL promoters. Optionally, further rounds of randomization and selection for cells showing increased signal-to-noise ratio for sensing the presence or absence of H₂ can be performed.

In another embodiment a method is provided for using the evolved HupSL promoter region and/or other evolved components of the H₂-sensing system as the H₂-sensing system used for evolution of the hydrogenase.

An exemplary method to evolve a H₂-sensing system comprises the following steps:
(a) providing a library of recombinant HupSL promoters and/or H₂-sensing proteins;
(b) transferring the recombinant HupSL promoters and/or H₂-sensing proteins to a pool of host microorganisms such that each host microorganism comprises one or more unique recombinant H₂-sensing systems, each host microorganism further comprising the lack of hydrogenases;
(c) culturing each of the host microorganisms under conditions where H₂ is not produced and H₂ is not supplied;
(d) measuring the level of the reporter gene product in vivo produced by each microorganism;
(e) comparing the level of reporter gene product produced by each recombinant H₂-sensing system; and,
(f) selecting those microorganisms with the lowest reporter gene product;
(g) culturing each of the selected host microorganisms under conditions where H₂ is supplied exogenously;
(h) measuring the level of the reporter gene product in vivo produced by each microorganism;
(i) comparing the level of reporter gene product by each recombinant H₂-sensing system;
(j) selecting those microorganisms with the highest reporter gene product;
(k) isolating the plasmid comprising the recombinant H₂-sensing systems from the selected microorganisms;
(l) randomizing and/or shuffling the recombinant H₂-sensing systems to generate a further library of the recombinant H₂-sensing systems; and
(m) iterating as necessary steps (b) through (l) and stopping at step (k) when the recombinant H₂-sensing systems fails to demonstrate further increased signal:noise ratio above the prior iteration.

An additional exemplary method for evolving a H₂-sensing system comprises the steps of: (1) providing DNA comprising a HupSL promoter coupled with a reporter gene; (2) transferring the DNA to a host microorganism lacking endogenous hydrogenase activity; (3) culturing the host microorganism under conditions where H₂ is not produced and H₂ is not added; (4) measuring the level of the reporter gene product in vivo; (5) selecting microorganisms with the lowest level of reporter gene product; (6) re-culturing those selected microorganisms having the lowest basal level of reporter gene product in the presence of H₂; and (7) selecting microorganisms with the highest level of reporter gene product. In the above exemplary methods, the signal:noise ratio of the H₂-sensing system is evolved if, in the absence of added H₂, the level of H₂ sensed is less than that sensed by the wild-type H₂-sensing system, and/or, in the presence of added H₂, the level of H₂ sensed is greater than that sensed by the wild-type H₂-sensing system.

In the exemplary methods above, the H₂-sensing system may comprise a mutagenized and/or shuffled HupSL promoter region or at least one mutagenized and/or shuffled H₂-sensing protein such as HupUV, HupT and/or HupR. In certain embodiments, the microorganisms may be cultured in the presence of O₂.

### Examples

The following examples are provided for illustrative purposes only and are not intended to limit the scope of the invention.

### Research Design and Methods

### Development of a High-Throughput H₂ Production Screening Assay

The lack of high-throughput screening methods for detecting hydrogenase activity has prevented the research community from selecting more diverse and efficient H₂-producing organisms or enzymes from large populations of candidates. The inventors have developed a new screening or selection technique, based on the H₂-sensing properties of the purple, non-sulfur photosynthetic bacterium R. capsulatus. This assay couples the H₂-sensor response of R. capsulatus to a reporter/selection gene fused to the promoter region that is normally up-regulated in response to the presence of H₂. In some embodiments, the production of H₂ will depend only on the expression and activity of recombinant [FeFe]-hydrogenases by using a R. capsulatus strain that lacks the native [NiFe]-hydrogenase activity. See, for example, Table 1.

### Platform Choice

The inventors have shown that C. acetobutylicum, C. pasteurianum and C. reinhardtii [FeFe]-hydrogenases can be functionally expressed in E. coli. The ease and knowledge of genetic manipulation in E. coli makes it an efficient platform for carrying out H₂-sensing and directed evolution. However, by observation: (1) the genetically complete H₂-sensing system of R. capsulatus at 24 kb is long and would be difficult to transfer to E. coli, and it is likely that the transcription level of each of the H₂-sensing components is finely tuned for efficient H₂ sensing in R. capsulatus; (2) the promoter regions of the genes would need to be engineered to function in E. coli (for instance, expression of the HupTUV operon in E. coli results in accumulation of only the HupT protein, when HupUV is expressed only HupU accumulates); (3) transcriptional activation of the HupSL promoter is functionally complex requiring the coordinated binding of R. capsulatus HupR, σ70-RNAP, and IHF which might not occur through substitution by E. coli transcriptional regulators. With these potential difficulties, the native H₂-sensing system of R. capsulatus is optimized first, and the desired [FeFe]-hydrogenases can be transferred into it, though it is understood that other microorganisms are similarly appropriate for utilizing the system described herein. As mentioned above, the promoter region of HupSL encoding the uptake hydrogenase in R. capsulatus is well detailed which will facilitate the creation of gene fusions for reporter assays. Also, mutants of R. capsulatus are available for each of the H₂-sensing system components, HupUV, HupR, HupT, and HupSL, as well as for the nitrogenase-negative strain RC18 which is incapable of producing H₂ due to a mutation in the Nif-18 gene.

Co-Expression of [FeFe]-Hydrogenase Structural and Maturation Proteins in a Strain of R. capsulatus that Lacks Native H₂-Uptake [NiFe]-Hydrogenase Activity

Strain JP91 of R. capsulatus, which has its structural HupSL genes inactivated by an insertion element is used to avoid loss of the H₂ signal due to uptake hydrogenase activity. Background H₂ production in R. capsulatus can be further minimized by growing the cultures in a medium containing NH₄Cl, where N₂ fixation and concomitant H₂ production through the nitrogenase enzyme doesn't occur.

The C. acetobutylicum HydA [FeFe]-hydrogenase and its three hydrogenase maturation proteins, HydE, HydF, and HydG, are each expressed behind Tn5α promoters. These are tested first since the functional expression of C. acetobutylicum [FeFe] hydrogenase has been well-studied in E. coli. Other [FeFe]-hydrogenases are tested as well. These include hydrogenases from C. reinhardtii (HYDA1 or HYDA2) which have also been successfully expressed as functional enzymes in E. coli, or the [FeFe]-hydrogenase systems from Shewanella oneidensis, Bacteroides thetaiodaomicron, or Desulfovibrio vulgaris, whose genomes contain putative [FeFe]-hydrogenase maturation and structural genes. The Tn5α promoter which drives constitutive transcription in R. capsulatus at reasonably high levels will regulate expression of the hydrogenases and their attendant maturation proteins. Alternatively, the puf promoter may also be used as it allows for fructose-inducible expression of genes, although at somewhat lower expression levels. The [FeFe]-hydrogenase structural and maturation genes can be introduced into R. capsulatus using two broad host range plasmids along with the HupSL::reporter fusion assembly. As with the E. coli system, an anoxic incubation period is carried out to allow assembly and stability of the newly expressed enzymes.

Detection of H₂-Production Activity by Coupling the R. capsulatus H₂-Sensing System to an Appropriate Reporter Gene

Initial work utilizes the lacZ system described by Colbeau and Vignais (Colbeau, A., and P. M. Vignais, Use of hupS::lacZ gene fusion to study regulation of hydrogenase expression in Rhodobacter capsulatus: stimulation by H2. J Bacteriol, 1992. 174(13): p. 4258-64, the contents of which are incorporated herein by reference), where H₂ production is coupled to the reduction of ONPG by β-galactosidase. However, ONPG is not permeable through R. capsulatus membranes. As such, the cells must be lysed in order to assay for H₂ production signal, making these fusions less amenable to high-throughput cell screening. Xgal can be used as a means of screening R. capsulatus colonies to measure the transcriptional activation of the fruP promoter in a fruB::lacZ fusion, however this screen is not amenable for high-throughput applications. Signaling mechanisms useful herein include the Covalys SNAP™ and Promega HaloTag™ systems. In these systems, the fused reporter protein catalyst converts a fluorescent cell-permeable compound into a fluorescent marker covalently bound to the catalyst. Between the two systems, 10 fluorescent dyes are available. These dyes are screened to assess which gives the best signal in R. capsulatus in terms of permeability and fluorescence properties, considering the background fluorescence of the native pigments. These systems allow a direct measure of H₂ production by individual cells. When tied in with FACS, about 1.1 million cells per hour can be partitioned based on a fluorescence cut-off. There are fluorogenic substrates of β-galactosidase, such as fluorescein di-b-D-galactopyranoside (FDG) that are useful for carrying out directed evolution using FACS. The permeability of these compounds to R. capsulatus will have to be tested. A more direct method of signaling would be to fuse green fluorescent protein (GFP) or related fluorescent proteins to the HupSL promoter. In this case, cell permeability of fluorescent probes would not be an issue. GFPs such as those from Evocatal, GMBH are know to be fluorescent both in the presence and absence of O₂ for fluorescence. Other signaling systems available include the use of antibiotic resistance genes such as the aadA gene for spectinomycin resistance, or the use of gain-of-function genes such as the fruA fructose permease gene. Both of these systems have the advantage of being selective and as such are not limited by the through-put rate of FACS. However, it would necessary to determine whether they would suitably distinguish between low and high levels of promoter upregulation.

### Coupling the H₂-Sensing Assay to Enrichment of Cell Populations for H₂ Production

Signaling systems for the assay (e.g. SNAP fluorescence, GFP, or antibiotic resistance) are verified for the ability to distinguish between cells that can and cannot produce H₂. The assay is challenged with cell populations containing various ratios of cells having active or non-active [FeFe]-hydrogenase in order to determine the degree of enrichment for each particular signaling system.

Validation of the Assay by Screening Hydrogenases Derived from Directed Evolution

Directed evolution of proteins is used to create diversity in catalysts and to yield proteins with improved functions. Directed evolution involves two principal steps, (1) randomization/shuffling of the protein coding sequence and (2) screening for activity, with successful protein sequences being used as templates for the next round of randomization and screening. Nagy et al. describe the expression of active, shuffled [FeFe]-hydrogenases in a heterologous E. coli-expression system. The high-throughput H₂-sensing assay can be validated by screening such randomized pools of [FeFe]-hydrogenases for enzymes with improved activity. The two RegA (redox inhibition) binding sites of the hupSL promoter can be modified to regulate HupR activation.

### Experimental Protocol

### Development of the High-Throughput Assay

### (a) Strains, Plasmids and Growth Conditions

TABLE 1
Strains used to develop assay.

| Strain, plasmid, | Genotype or | Reference or |
|---|---|---|
| or gene | phenotype | description |
| R. capsulatus | | |
| strains | | |
| B10 | Parent Wild type | ATCC |
| RC18 | Nif-18 | No nitrogenase activity ^{a} |
| JP91 | hupSL | No uptake hydrogenase ^{a} |
| VBC1 | hupR | No regulator protein ^{a} |
| BSE8 | hupT | No histidine kinase ^{a} |
| JBC11B | hupUV | No H2-sensor protein ^{a} |
| E. coli strains | | |
| DH5α | For plasmid construction | GIBCO-BRL |
| ATCC824 | C. acetobutylicum | ATCC |
| | hyd genes | |
| XL10-gold KanR | Contains the aph promoter | Stratagene |
| C. reinhardtii | | |
| strains | | |
| CC124 | C. reinhardtii hyd genes | Chlamydomonas Center |
| | | culture collection |
| Plasmids | | |
| pAC142 | hupSL::lacZ, Tcr | B-galactosidase fusion ^{a} |
| pPHU281 | Suicide plasmid, Tcr | For triparental mating ^{a} |
| pRK2013 | Mobilizing plasmid, | For triparental mating ^{a} |
| | pRK2013, Kmr | |
| pBBR1MCS-1 | Broad host range, Cmr | ^{b} |
| pBBR1MCS-2 | Broad host range, Kmr | ^{b} |
| pBBR1MCS-3 | Broad host range, Tcr | ^{b} |
| pBBR1MCS-5 | Broad host range, Gmr | ^{b} |
| pGlow-Bs2 | O₂-insensitive GFP | Evocatal |
| pGlow-Pp1 | O₂-insensitive GFP | Evocatal |
| pSS26b | SNAP fusion sequence | Covalys |
| pHT2 | HaloTag fusion sequence | Promega |

^{a} Colbeau and Vignais;
^{b} Kovach, et al., Four new derivatives of the broad-host-range cloning vector pBBR1MCS, carrying different antibiotic-resistance cassettes. Gene, 1995. 166(1): p. 175-6.

All of the R. capsulatus strains are grown in mineral RCV medium plus 30 mM DL-malate as the C source as per Colbeau and Vignais (see citation above). The N source is either 7 L-glutamate (MG medium, nitrogen fixing, H₂ evolving), or 7 mM ammonium sulfate (MN medium, non H₂-evolving medium). Growth is carried out at 30° C. either anaerobically under light (−2,500 lux in completely filled screw-cap tubes, or Petri plates under argon) or under dark conditions (aerobic, culture flasks filled to 20% capacity in a shaker at 200 RPM or Petri plates). Plasmid DNA can be transferred into R. capsulatus using tri- or di-parental mating. Antibiotic levels are: 5 μg/ml kanamycin, 3 μg/ml tetracycline, 3 μg/ml gentamicin, or 10 μg/ml spectinomycin. However, all plasmid transfer methods are contemplated herein.

(b) Cloning of [FeFe]-Hydrogenases into R. capsulatus

DNA manipulation, including DNA purification, PCR amplification, reverse transcription, and cloning are carried out essentially as per as per Sambrook and Russell (Molecular cloning: a laboratory manual. 3rd ed. 2001, Cold Spring Harbor, N.Y.: Cold Spring Harbor Laboratory Press). DNA for the HydA, HydE, HydF, and HydG genes of C. acetobutylicum is PCR amplified as per King, et al. (Functional studies of [FeFe] hydrogenase maturation in an Escherichia coli biosynthetic system. J Bacteriol, 2006. 188(6): p. 2163-72) using KOD polymerase (Novagen). Each of these genes can be placed behind a separate constitutive Tn5 aph kanamycin promoter. The aph promoter is generated from the native aph gene in Stratagene XL10-gold KanR cells using a 5′ PCR primer that includes a KpnI restriction site, and one of three 3′ primers for inclusion of restriction sites for NdeI, PciI, or NcoI. These primers connect the 3′ end of the aph promoter to the starting codons of HydA and HydF (NdeI), HydG (PciI), and HydE (NcoI). In this way, the separate genes are accessible as cassettes for future directed-evolution experiments. The HydA and HydG genes are introduced into pBBR1MCS-5 (Gmr) as plasmid pCahydAG of size 8.3 Kb. Similarly, HydF and HydE are cloned into vector pBBR1MCS-2 (Kmr) as plasmid pCahydEF of size 7.9 Kb. As a negative control, plasmid pCahydE lacks the HydF gene. This plasmid is used as a negative control for determining which HupS::fusion system gives the cleanest signal-to-noise ratio. The above plasmids along with the H₂-sensing HupS::lacZ fusion plasmid pAC142, (Tcr) are sequentially transformed into the host JP91 HupSL(−) strain.

### (c) High-Throughput Constructs

Initial experiments will establish the relative permeability and signal of the five HaloTag and five SNAP fluorescent dyes. The HaloTag and SNAP genes will each be fused behind an aph promoter and delivered in a pBBR1MCS vector. Cells are exposed for 2 hours to 2.5 μM dye prior to washing. As a control, one group of cells is pre-blocked with a non-fluorescent reactive species to tie up the active site of the tagging enzyme. Cells are monitored using fluorescence spectroscopy and FACS, with the excitation laser and emission filters appropriate to the dye. Cell permeation for the best fluorescent dyes will be optimized. The system of choice is then be fused behind the hupSL promoter in place of lacZ as described above. As necessary, GFP or gain-of-function (fructose permease or spectinomycin resistance) genes are tested for improved high-throughput signaling of H₂ production, using fusion of the respective genes behind the hupSL promoter.

### (d) Hydrogenase Activity Assays

H₂ production by [FeFe]-hydrogenases in R. capsulatus will initially be detected by gas chromatographic headspace analysis of the following four R. capsulatus strains: B10 (wild type), JP91 (HupSL-), and JP91 phupSL::lacZ with active (+) or inactive (−) [FeFe]-hydrogenase constructs (where the inactive construct lacks HydF). Cells are grown to mid-log phase in the dark under anerobic conditions in MN medium supplemented with 500 μM Fe-citrate and the appropriate antibiotics. Under these conditions, nitrogenases do not fix nitrogen and therefore do not produce H₂. The only H₂ produced should be the result of the heterologously expressed [FeFe]-hydrogenases. As necessary, other heterologous [FeFe]-hydrogenase systems from C. reinhardtii, Shewanella oneidensis, Bacteroides thetaiodaomicron, and Desulfovibrio vulgaris can be incorporated and tested for H₂ production. To make sure the H₂-sensing system is functional, β-galactosidase activity is compared among the four R. capsulatus strains following the assay of Colbeau and Vignais. The H₂-sensing system is then be modified to couple the sensing of H₂ to the chosen high-throughput construct, e.g. the hupSL::SNAP system.

### (e) Selection Systems

JP91 strains containing active or inactive [FeFe]-hydrogenase constructs and the chosen signaling system are used to optimize FACS signal-to-noise ratio and cell separation techniques. Experimental parameters include the culture age, fluorescent dye concentration, and dye reaction time, flow rate and concentration of cells through the FACS, as well as FACS threshold and instrument settings. Using these FACS parameters, a mock selection is carried out on cultures in which [FeFe]-hydrogenase (+) cells will be mixed in increasingly smaller ratios into larger populations of [FeFe]-hydrogenase (−) cells (e.g. 1:10, 1:100, up to 1:10⁸). Population dynamics is followed by quantitative PCR of the HydF gene in comparison to the control, HydE. These data can be used to model enrichment scenarios for directed evolution experiments.

### (f) Directed Evolution of Hydrogenases

The shuffled [FeFe]-hydrogenase library described in Nagy et al. is inserted into the hydA cassette of the H₂-sensing system and put through FACS for either a representational analysis of H₂ production by members of the library or for the selection of cells producing H₂. Cell populations screened for medium-to-high H₂ production are carried forward in directed evolution (less stringent selection during early rounds of protein shuffling experiments adds to the complexity of the library). Further evolution at each round includes: (a) reshuffling of the enriched population's HydA libraries selected from the previous round; (b) cloning into original (non-selected) vectors and cell lines (this limits the non-intended selection of such attributes as a permissively upregulated H₂ sensing system, or a permissive promoter region); and, (c) FACS selection, which typically involves some increase in stringency each round, e.g. the use of increasingly stringent FACS cut-off values to select for more highly fluorescent cells, or the use of increased levels of O₂ exposure during the pre-assay phase in order to select for O₂-tolerant [FeFe]-hydrogenases. Cycles can be carried out iteratively until no further increase in H₂ production or O₂ tolerance is noted in comparison to the previously enriched pool of cells. The final selected cell pool is plated, and individual HydA genes sequenced from ˜100 colonies in order to determine the extent of evolution and divergence between the HydA genes. Representative clones from the families of evolved HydA sequences are characterized for H₂-production or O₂-tolerance levels in R. capsulatus and compared to those levels measured in the initial HydA strain. This comparison is made using both gas chromatography of the headspace gas and fluorescence spectroscopy.

A number of patents, patent application publications, and scientific publications are cited throughout and/or listed at the end of the description. Each of these are incorporated herein by reference in their entirety. Likewise, all publications mentioned in an incorporated publication are incorporated by reference in their entirety.

Examples in cited publications and limitations related therewith are intended to be illustrative and not exclusive. Other limitations of the cited publications will become apparent to those of skill in the art upon a reading of the specification and a study of the drawings.

The words “comprise”, “comprises”, and “comprising” are to be interpreted inclusively rather than exclusively.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A method for measuring H₂ production by a hydrogenase comprising:
providing a foreign DNA comprising a HupSL promoter coupled with a reporter gene;
transferring the foreign DNA to a host microorganism lacking endogenous hydrogenase activity;
transferring to the host microorganism one or more plasmids comprising genes encoding a recombinant hydrogenase;
culturing the host microorganism under conditions permissive to production of H₂ from the recombinant hydrogenase; and
measuring the level of the reporter gene product in vivo;
wherein production of H₂ by the hydrogenase activates the HupSL promoter and the reporter gene product increases.

2. A method for evolving a hydrogenase comprising:
providing DNA comprising a HupSL promoter coupled with a reporter gene;
transferring the DNA to a host microorganism lacking endogenous hydrogenase activity;
transferring to the host microorganism one or more plasmids comprising a recombinant hydrogenase and associated assembly proteins;
culturing the host microorganism under conditions permissive to production of H₂ from the recombinant hydrogenase;
measuring the level of the reporter gene product in vivo;
comparing the level of production of H₂ by the recombinant hydrogenase to the level of H₂ produced by a wild-type hydrogenase cultured under the same conditions; and
selecting a microorganism with increased H₂ production over the H₂ production by a microorganism having a wild-type hydrogenase;
wherein production of H₂ by the hydrogenase activates the hupSL promoter and the reporter gene product increases; and
wherein the recombinant hydrogenase is evolved if the level of H₂ produced is greater than that produced by the wild-type hydrogenase.

3. The method of claim 1 wherein the reporter gene is Green Fluorescent Protein, HaloTag, SNAP, spectinomycin resistance, or any other antibiotic resistance gene.

4. The method of claim 1, wherein the foreign DNA is selected from the group consisting of a plasmid, cosmid, insertion element, transposon, chromosome, and naked DNA.

5. The method of claim 1 wherein the host microorganism contains one or more disrupted, inactive endogenous hydrogenases.

6. The method of claim 1 wherein the recombinant hydrogenase is an [Fe—Fe] hydrogenase, a [Ni—Fe] hydrogenase, an Fe—S free hydrogenase, or a nitrogenase.

7. The method of claim 6 wherein the hydrogenase is generated by mutagenizing, shuffling, or mutagenizing and shuffling hydrogenase genes to provide a library of hydrogenases.

8. The method of claim 1 wherein the microorganism is any nitrogenase-containing organism.

9. The method of claim 1 wherein the microorganism is R. capsulatus.

10. The method of claim 1 wherein the recombinant hydrogenase is a nitrogenase.

11. The method of claim 1 wherein the one or more plasmids further comprise at least one associated assembly protein.

12. A plasmid comprising a hupSL promoter coupled with a fluorescence reporter gene.

13. A microorganism comprising:
a) a foreign DNA comprising a hupSL promoter coupled with a reporter gene, and
b) one or more plasmids comprising genes encoding a recombinant hydrogenase;
wherein the microorganism lacks endogenous hydrogenase activity.

14. The microorganism of claim 13, wherein the microorganism further comprises one or more plasmids comprising at least one gene encoding a hydrogenase assembly protein, ferredoxin, or a protein involved in the H₂-sensing apparatus.

15. A high-throughput assay for measuring in vivo H₂ production by a recombinant hydrogenase comprising:
a host microorganism, an H₂ sensor, a recombinant hydrogenase, and a HupSL promoter coupled with a reporter gene, and
wherein the host microorganism lacks endogenous hydrogenase activity.

16. The assay of claim 15, wherein the recombinant hydrogenase is an [Fe—Fe] hydrogenase, a [Ni—Fe] hydrogenase, an Fe—S free hydrogenase, or a nitrogenase.

17. The assay of claim 15, wherein the reporter gene is Green Fluorescent Protein, HaloTag, SNAP, spectinomycin resistance, or any other antibiotic resistance gene.

18. The assay of claim 15, wherein the H₂ sensor is HupUV.

19. The assay of claim 15, wherein the host microorganism is cultured under oxygen or carbon monoxide levels inhibitory to H₂ production by a wild-type hydrogenase.

20. A method to evolve a hydrogenase comprising:
(a) providing a library of recombinant hydrogenases;
(b) transferring the recombinant hydrogenases to a pool of host microorganisms such that each host microorganism comprises one or more unique recombinant hydrogenases, each host microorganism further comprising hydrogenase support proteins, an H₂ sensor, and a hupSL promoter coupled with a reporter gene;
(c) culturing each of the host microorganisms under conditions permissive to production of H₂ from the recombinant hydrogenases;
(d) measuring the level of the reporter gene product in vivo produced by each microorganism;
(e) comparing the level of production of H₂ by each recombinant hydrogenase to the level of H₂ produced by a wild-type hydrogenase cultured under the same conditions; and
(f) selecting a microorganism with increased H₂ production over the H₂ production by a microorganism having a wild-type hydrogenase;
(g) isolating the plasmid comprising the recombinant hydrogenase from the selected microorganism;
(h) randomizing and/or shuffling the hydrogenase to generate a further library of recombinant hydrogenases; and
(i) iterating as necessary steps (b) through (h) and stopping at step (g) when the recombinant hydrogenase fails to demonstrate further increased H₂ production above the prior iteration;
wherein production of H₂ by the hydrogenase activates the hupSL promoter and the reporter gene product increases.

21. A method to identify an oxygen-resistant or a carbon dioxide-resistant hydrogenase, the method comprising:
providing a foreign DNA comprising a hupSL promoter coupled with a reporter gene;
transferring the foreign DNA to a host microorganism lacking endogenous hydrogenase activity and comprising a recombinant hydrogenase;
culturing the host microorganism in the presence of oxygen or carbon monoxide levels inhibitory to H₂ production by a wild-type hydrogenase;
measuring the level of the reporter gene product in vivo; and
comparing the level of production of H₂ by the recombinant hydrogenase to the level of H₂ produced by the wild-type hydrogenase cultured under the same conditions;
wherein production of H₂ by the hydrogenase activates the hupSL promoter and the reporter gene product increases; and
wherein the recombinant hydrogenase is oxygen-resistant or carbon dioxide-resistant if the level of H₂ produced is greater than that produced by the wild-type hydrogenase.

22. A method to evolve a H₂-sensing system comprising:
(a) providing a library of recombinant HupSL promoters and/or H₂-sensing proteins;
(b) transferring the recombinant HupSL promoters and/or H₂-sensing proteins to a pool of host microorganisms such that each host microorganism comprises one or more unique recombinant H₂-sensing systems, each host microorganism further comprising the lack of hydrogenases;
(c) culturing each of the host microorganisms under conditions where H₂ is not produced and H₂ is not supplied;
(d) measuring the level of the reporter gene product in vivo produced by each microorganism;
(e) comparing the level of reporter gene product produced by each recombinant H₂-sensing system; and,
(f) selecting those microorganisms with the lowest reporter gene product;
(g) re-culturing each of the selected host microorganisms under conditions where H₂ is supplied exogenously;
(h) re-measuring the level of the reporter gene product in vivo produced by each microorganism;
(i) comparing the level of reporter gene product by each recombinant H₂-sensing system; and,
(j) selecting those microorganisms with the highest reporter gene product;
(k) isolating the plasmid comprising the recombinant H₂-sensing systems from the selected microorganisms;
(l) randomizing and/or shuffling the recombinant H₂-sensing systems to generate a further library of the recombinant H₂-sensing systems; and
(m) iterating as necessary steps (b) through (l) and stopping at step (k) when the recombinant H₂-sensing systems fails to demonstrate further increased signal:noise ratio above the prior iteration.

23. A method for evolving a H₂-sensing system comprising:
providing DNA comprising a HupSL promoter coupled with a reporter gene;
transferring the DNA to a host microorganism lacking endogenous hydrogenase activity;
culturing the host microorganism under conditions where H₂ is not produced and H₂ is not added;
measuring the level of the reporter gene product in vivo;
selecting microorganisms with the lowest level of reporter gene product;
re-culturing those selected microorganisms having the lowest basal level of reporter gene product in the presence of H₂;
selecting microorganisms with the highest level of reporter gene product;
wherein the signal:noise ratio of the H₂-sensing system is evolved if, in the absence of added H₂, the level of H₂ sensed is less than that sensed by the wild-type H₂-sensing system, and/or, in the presence of added H₂, the level of H₂ sensed is greater than that sensed by the wild-type H₂-sensing system.

24. The method of claim 23, wherein the HupSL promoter region is mutagenized, shuffled, or mutagenized and shuffled.

25. The method of claim 23, wherein the H₂-sensing system comprises at least one mutagenized, shuffled, or mutagenized and shuffled H₂-sensing proteins selected from HupUV, HupT and HupR.

26. The method of claim 23, wherein the microorganisms are cultured in the presence of O₂.


SEQUENCE LISTING (1 sequence)

SEQ ID NO: 1 - DNA, 532 bp, Rhodobacter capsulatus
ggatctgccctgcgtcggcggggcgatcgacccggcgccgctgtttcaacttctggcggcaggcgagcgccccgaccgtgtcgcccatgcgctgcacgccagccttgcgcaggcttttgccgccgaagcgcggcggctgatcgaagccggacaggccgaggcggtggcgctgacgggcggctgttttcagaattccggacttgcaaccatgacgcgaaatttccttgccgatcaaggcattctgacgcagggccgcatcccggccaacgatgggggccttgccctgggtcaggcccttgtcgcggcggcaaaattggaaagcaactgaccgaagtggaaatggaatcggtcaaaatactttcggtttttttacgtgacaaaatcacacaccattgaaaaacaaggaaaaatttttctgcgacaatttgccgtttctcattggcggaaaccggcccaaccatgagaattccactctacccatgggcgcgccgaggaggtcacggcgcgtcacaaggcaagggagggacaattg